(19) United States
(12) Patent Application Publication
(10) Pub. No.: US 2011/0123575 A1
(43) Pub. Date: May 26, 2011

(54) MODIFIED RELEASE NIACIN FORMULATIONS

(75) Inventors: Rajesh Vooturi, Hyderabad (IN); Dhanajay Singare, Jalna (IN); Shantanu Yeshwant Damle, Thane (IN); Pradeep Jairao Karatgi, Hyderabad (IN); Sesha Sai Marella, Guntur (IN); Harshal Prabhakar Bhagwatwar, Hyderabad (IN); Ish Kumar Khanna, Alpharetta, GA (US); Raviraj Sukumar Pillai, Hyderabad (IN)

(73) Assignees: DR. REDDY'S LABORATORIES LTD., Hyderabad, 500 016, Andhra Pradesh (IN); DR. REDDY'S LABORATORIES, INC., Bridgewater, NJ (US)

(21) Appl. No.: 12/995,718

(22) PCT Filed: Jun. 2, 2009
(86) PCT No.: PCT/US09/45927

§ 371 Date: Dec. 2, 2010

(30) Foreign Application Priority Data
Jun. 2, 2008 (IN) 1345/CHE/2008
May 18, 2009 (IN) 1145/CHE/2009

(51) Int. Cl. A61K 9/00; A61K 31/4406; A61P 3/06; A61P 9/10

(52) U.S. Cl. 424/400; 514/356

(57) ABSTRACT

Modified release pharmaceutical formulations comprising niacin in a non-swellable core, and processes for preparation of the formulations.

### INTRODUCTION

Aspects of the present invention relate to pharmaceutical formulations comprising niacin in modified, including extended, delayed, and delayed-extended, release forms for oral administration. Methods of using the formulations of the invention to modulate niacin-induced flushing and hepatotoxicity are also included.

Dyslipidemia is elevation of plasma cholesterol and/or triglycerides (TG) or a low high density lipoprotein (HDL) level that contributes to the development of atherosclerosis. Causes may be primary (genetic) or secondary. Diagnosis is by measuring plasma levels of total cholesterol, TG, and individual lipoproteins. Treatment is dietary changes, exercise, and lipid-lowering drugs. Diabetes is an especially significant secondary cause because patients tend to have an atherogenic combination of high TG; high small, dense LDL fractions; and low HDL (diabetic dyslipidemia, hypertriglyceridemic hyperapo B). Patients with type 2 diabetes are especially at risk. The combination may be a consequence of obesity and/or poor control of diabetes, which may increase circulating free fatty acids (FFA), leading to increased hepatic very low density lipoprotein (VLDL) cholesterol production. TG-rich VLDL then transfers TG and cholesterol to low density lipoprotein (LDL) and HDL, promoting formation of TG-rich, small, dense LDL and clearance of TG-rich HDL. Diabetic dyslipidemia is often exacerbated by the increased caloric intake and physical inactivity that characterize the lifestyles of some patients with type 2 diabetes. Women with diabetes may be at special risk for cardiac disease from this form.

Niacin also called nicotinic acid or 3-pyridinecarboxylic acid) is a white, crystalline powder, very soluble in water, having structural Formula I.

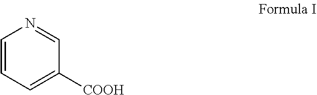

Niacin, when taken in large doses, has been shown to reduce levels of total cholesterol (TC), LDL and TG. It has also been shown to increase HDL levels in circulation and reduce cardiovascular risk in patients with documented cardiovascular disease. Multiple mechanisms have been proposed for the lipid modulating effects of niacin. It blocks or inhibits lipolysis in adipose tissue thus reducing free fatty acids in plasma. Niacin inhibits uptake of apolipoprotein A1 (apoA1) by the liver without affecting the clearance of cholesterol associated with HDL.

Commercially, niacin is available in immediate release (IR) formulations as well as sustained release (SR) formulations and intermediate release formulations. Niacin IR is generally more effective than other niacin products for increasing HDL-C (high density lipoprotein-cholesterol). The extent of the lipid reduction varies due to differing baseline levels. Niacin IR therapy should be initiated slowly, with a maximum daily dose of 3 grams. The following description highlights various IR and SR products available commercially along with their doses and efficacy in the treatment of dyslipidemia, the information being based on the report of the Niacin Product Selection Workgroup titled “Veterans Administration Niacin Product Selection,” United States Department of Veterans Affairs, Veteran Health Administration, Aug. 31, 1999 (obtained at the web site www.pbm.va.gov):

Niacin (using a nonprescription IR product, Rugby Laboratories) at a dose of 2-3 grams/day decreased LDL-C by 16-22% and TG by 39-42%, and increased HDL-C by 31-35%.

Niacin (using a prescription IR product NICOLAR™, Rhone-Poulenc Rorer) at a dose up to 3 grams/day decreased LDL-C by 28% and TG by 38%, and increased HDL-C by 22%. In an additional study, NICOLAR at a dose up to 3 grams/day decreased LDL-C by 25% and TG by 26%, and increased HDL-C by 36%. In a third study, NICOLAR at an average dose of 2.25 grams/day decreased LDL-C by 16% and TG by 29%, and increased HDL-C by 27%.

Niacin (using a nonprescription IR product, Goldline Laboratories) at a dose of 3 grams/day decreased LDL-C by 2% and TG by 29%, and increased HDL-C by 25%.

Niacin (using a Kos Pharmaceuticals IR product manufactured for research purposes only) at a dose of 1.5-3 grams/day decreased LDL-C by 13-21% and TG by 19-24%, and increased HDL-C by 10-24%.

Commercial niacin SR products are available as nonprescription products as well as by prescription.

Niacin SR is generally more effective at decreasing LDL-C than niacin IR, although less effective at increasing HDL-C. Niacin SR should be initiated at approximately one-half of the niacin IR dose; the maximum daily dose is 2 grams.

Niacin (using a nonprescription SR product, Goldline Laboratories) at a dose of 1.5-2 grams/day reduced LDL-C by 22%-33% and TG by 25-30%, and increased HDL-C by 13%-17%.

Niacin (using a nonprescription SR product NICOBID™, Armour Pharmaceuticals) at a dose of 3 grams/day reduced LDLC by 17% and TG by 2%, and increased HDL-C by 8%. In another study, NICOBID at a dose of 1-2 grams niacin/day reduced LDL-C by 16% and TG by 11%, and increased HDL-C by 12%.

Niacin (using a nonprescription SR product SLO-NIACIN™, Upsher-Smith) at a dose of grams/day reduced LDL-C by 18% and TG by 29%, and increased HDL-C by 16%. In a retrospective study, SLO-NIACIN at an average daily dose of 1.5 grams of niacin, reduced LDL-C by 24% and TG by 33%, and increased HDL-C by 6%.

Niacin (using a nonprescription SR product ENDUR-ACIN™, Endurance Products Corporation) at a dose of 1.5 grams/day reduced LDL by 16%, and increased TG by 4% and HDL-C by <1%. In another study, ENDUR-ACIN at a dose of 1.5-2 grams/day reduced LDL-C by 15-22% and TG by 10-25%, and increased HDL-C by 9-16%. The same study examined differences between younger (20-49 years old) and older (50-70 year old) patients. At a dose of 1.5-2 grams/day, LDL-C was reduced by 29% and TG by 21%, and HDL-C was increased by 8% in older patients; in younger patients LDL-C was reduced by 16%, TG increased by <1% and HDLC increased by 7%. In a third study, ENDUR-ACIN at a dose of 1.5-2 grams/day reduced LDL-C by 20-26% and TG by 9-11° A, and increased HDL-C by 4-9%.

Niacin (using a nonprescription SR product, Rugby Inc.) at a dose of 1.2 grams/day reduced LDL-C 6%, and increased TG 11% and HDL-C 2%.

Niacin in the form of a prescription intermediate release product NIASPAN™, Kos Pharmaceuticals, has equivalent daily dosing and similar efficacy to niacin IR. Patients should initiate therapy with the dosing starter pack (Niaspan 375 mg, 500 mg, and 750 mg tablets, each at bedtime for one week and increase 500 mg in a four week period).

NIASPAN, at a dose of 1.5 grams niacin at bedtime reduced LDL-C by 13% and TG by 10%, and increased HDL-C by 19%. In a second study, Niaspan up to 3 grams niacin at bedtime reduced LDL-C by 18% and TG by 26%, and increased HDL-C by 32%. A third study examined Niaspan at a dose of 1-2 grams niacin/day at bedtime, reducing LDL-C by 6-15% and TG by 21-28%, and increasing HDL-C by 17-23%.

NIASPAN is indicated as an adjunct to diet for the reduction of elevated TC, LDL-C, apolipoprotein B and TG levels, and to increase HDL in patients with primary hypercholesterolemia and mixed dyslipidemia. NIASPAN® is also indicated to reduce the risk of recurrent nonfatal myocardial infarction and to slow the progression or promote the regression of atherosclerotic disease. NIASPAN is to be taken at bedtime, after a low-fat snack, and doses are individualized according to patient response.

By lowering VLDL levels, niacin also increases the level of HDL in the blood and therefore it is often prescribed for the patients with low HDL, who are also at a high risk of heart attack.

High doses of niacin have been shown to elevate fasting blood sugar levels, thereby worsening type 2 diabetes. Accordingly, niacin is contraindicated for persons with type 2 diabetes. The mechanism behind niacin-induced insulin resistance and diabetes is presently unknown.

U.S. Pat. Nos. 5,126,145, 5,268,181, 6,080,428, 6,129,930, 6,406,715, 6,469,035, 6,676,967, 6,746,691, 6,818,229, and 7,011,848 disclose sustained release formulations of nicotinic acid. U.S. Pat. No. 5,981,555, U.S. Patent Application Publication Nos. 2004/0053975 and 2005/0148556, and International Application Publication Nos. WO 2004/103370, WO 2006/017354, WO 2007/041499, WO 2004/111047, and WO 2009/005803 describe methods of reducing niacin-induced flushing.

Most of the documents and reports available in the literature indicate a pressing need for attempts to minimize flushing caused by niacin therapy. Though the introduction of NIASPAN into the market addressed some of the concerns with prior niacin therapy, in placebo-controlled clinical trials for NIASPAN, flushing episodes, i.e., warmth, redness, itching and/or tingling, were the most common treatment emergent adverse events reported by as many as 88% of patients, with 47% of the patients dropping out of the study due to flushing (source—summary basis of approval for NIASPAN).

A need exists for niacin-containing formulations that reduce the incidence of flushing, while providing benefits equivalent to commercially available formulations and little or no hepatotoxicity.

### SUMMARY

Aspects of the present invention relate to pharmaceutical formulations comprising modified (e.g., delayed, extended, and delayed-extended) release niacin for oral administration.

In an embodiment, the invention provides pharmaceutical formulations for the modified release of niacin comprising:

(a) a niacin-containing core comprising a therapeutically effective amount of niacin, a salt thereof, or a niacin prodrug, a pharmaceutically acceptable release controlling agent, and another pharmaceutically acceptable excipient

(b) optionally, a barrier coating layered onto the niacin-containing core; and

(c) optionally, an enteric coating applied directly onto either the core, if (b) is not present, or onto the barrier coating.

In an embodiment, the invention provides pharmaceutical formulations for the modified release of niacin, including a niacin-containing core comprising a therapeutically effective amount of niacin, a salt thereof, or a niacin prodrug, a pharmaceutically acceptable release controlling agent, and another pharmaceutically acceptable excipient.

In an embodiment, the invention includes pharmaceutical formulations for the modified release of niacin, comprising:

(a) a niacin-containing core comprising a therapeutically effective amount of niacin, a salt thereof, or a niacin prodrug, and a pharmaceutically acceptable excipient;

(b) optionally, a barrier coating layered onto the core; and

(c) optionally, an enteric coating applied directly onto the core, if (b) is not present, or onto the barrier coating.

In an embodiment, there are provided formulations comprising modified release niacin, the formulations providing statistically significant increases in plasma niacin levels (i.e., Cₘₐₓ and/or AUC), as compared to those obtained after oral administration of a similar amount of niacin from the commercially available NIASPAN intermediate release niacin product.

In an embodiment, there are provided formulations comprising modified release niacin, the formulations providing at least a two-fold increase in Cₘₐₓ and AUC, as compared to those obtained after oral administration of a similar amount of niacin from the commercially available NIASPAN intermediate release niacin product.

In an embodiment, there are provided formulations comprising modified release niacin, the formulations providing statistically reduced flushing, as compared to the flushing produced after oral administration of a similar amount of niacin from the commercially available NIASPAN intermediate release niacin product.

The presence of the barrier coating, applied between the niacin-containing core and the enteric coating, is an embodiment of the invention that can provide significantly higher systemic exposure of the niacin, upon administration to a mammal in need of administration of niacin.

In certain embodiments, the modified release formulations release their contained niacin at a slower rate into an aqueous fluid than is obtained with an immediate release formulation, but at a faster rate than is obtained with intermediate release and sustained release formulations known in the art, when tested under similar dissolution conditions.

In other embodiments, modified release formulations provide an in vitro release of their contained niacin at rates substantially equivalent to prior art formulations, when tested under similar dissolution conditions.

In certain embodiments, modified release formulations release niacin at a slower rate, and/or with a release delayed for a time, such as about the first 60 minutes or 120 minutes after oral dosing, which allow the simultaneous administration of anti-flushing agents to help control the flushing caused by niacin. For example, the present compositions allow for the simultaneous administration of flush-inhibiting agents such as non-steroidal anti-inflammatory agents (NSAIDs), cyclooxygenase-2 inhibitors, PGD2-antagonists, or other compounds with similar activity together with the modified release niacin formulation. The provision of slower and/or delayed release of the niacin with a co-administration or simultaneous immediate release flush-inhibiting agent allows for levels of the flush-inhibiting agent to build up in the body before peak concentrations of niacin are obtained. Subsequent extended release, provided by the release controlling substances in the niacin containing core, allow for sustained high levels of the niacin in the body.

In embodiments, less than about 50%, or less than about 25%, or less than about 10%, of contained niacin will be released into an aqueous medium having pH values less than about 4, within about 2 hours following immersion of a dosage form in the medium.

Processes for manufacturing the formulations of the invention as well as methods of using the formulations for the treatment of a variety of disease conditions are also described herein.

### BRIEF DESCRIPTION OF THE DRAWINGS

FIG. 1 is a graph illustrating in vitro dissolution profiles of products from Examples 1 and 2.

FIG. 2 is a graph illustrating in vitro dissolution profiles of products from Examples 3, 4, 5, 6, 7, and 8.

FIG. 3 is a graph illustrating the in vitro dissolution profile of a product from Example 9.

FIG. 4 is a graph illustrating in vitro dissolution profiles of products from Examples 10 and 11, NIACOR 500 mg tablets, and NIASPAN 500 mg tablets.

### DETAILED DESCRIPTION

The present invention relates to pharmaceutical formulations comprising modified (delayed, extended or delayed-extended) release niacin for oral administration.

The pharmaceutical formulations of the present invention with their unique in vitro and in vivo release profiles can provide effective niacin therapy with the benefits of reduced flushing, when compared with prior niacin formulations.

The term “niacin” is intended to include niacin free acid, any of its pharmaceutically acceptable salts, solvates, hydrates, polymorphs, a niacin prodrug, and mixtures thereof. As used herein, the term “prodrug” encompasses compounds other than niacin, which the body metabolizes to yield niacin, thus producing the same effect as described herein. Prodrug compounds specifically include, but are not limited to, nicotinamide, nicotinyl alcohol tartrate, d-glucitol hexanicotinate, aluminum nicotinate, niceritrol, and d,1-alpha-tocopheryl nicotinate.

“Formulation” in the context of present invention refers to a unit dose pharmaceutical formulation comprising niacin that is a solid dosage form, examples of solid dosage forms including, but not limited to, monolithic tablets, bilayered or multi layered tablets, capsules, tablets in capsules, and pellets, granules, or particles filled into capsules or compressed into tablets.

“Modified release” is intended to mean formulations which provide slow, delayed, extended or delayed-extended release of niacin, without limitation. Any mechanism of providing delayed, extended or delayed-extended release is included within the scope of the invention as long as the basic principles of the invention are met.

Formulations comprising niacin as described herein can provide one or more of the following advantages over the prior art formulations:

a) Significant reduction in niacin-induced flushing relative to immediate release niacin formulations.

b) Equivalent or reduced niacin-induced hepatotoxicity, when compared with the known sustained release formulations, or comparable with that of NIASPAN tablets of similar strength.

c) Improved dyslipidemia or anti-atherosclerotic profiles, as compared to other formulations.

Formulations comprising niacin as described herein can provide one or more of the following advantages over immediate release niacin formulations:

a) Significant reduction in niacin-induced flushing when compared with an immediate release composition at same dose.

b) Sustained drug levels over an extended period, and a potential for once- or twice-daily dosing.

c) Improved or comparable efficacy for dyslipidemic patients when compared with a presently commercially available composition at the same dose.

Formulations comprising niacin as described herein can provide one or more of the following advantages over sustained release niacin formulations:

a) Significant reduction in niacin-induced hepatotoxicity when compared with a presently commercially available sustained release composition at the same dose.

b) Improved efficacy for dyslipidemic patients when compared with a presently commercially available sustained release and immediate release compositions at the same dose.

Formulations comprising niacin as described herein can provide one or more of the following advantages over an intermediate release formulation such as the NIASPAN product:

a) Improved plasma niacin concentrations (AUC and/or Cₘₐₓ) after administering comparable doses.

b) Comparable or reduced flushing at comparable plasma niacin concentrations (Cₘₐₓ and/or AUC).

c) Comparable or enhanced efficacy at similar doses.

The term “modified release” is intended to include slow release, extended release, delayed release, controlled release, programmed release, pulsed release, a combination of delayed and extended release, and any other combination of two or more of the above release types excluding the intermediate release profiles of NIASPAN® marketed by Abbott.

In an embodiment of the invention, pharmaceutical formulations for the modified release of niacin comprise:

(a) a niacin-containing core comprising a therapeutically effective amount of niacin, a salt thereof, or a niacin prodrug, a pharmaceutically acceptable release controlling agent, and one or more other pharmaceutically acceptable excipients;

(b) optionally, a barrier coating applied onto the core; and

(c) optionally, an enteric coating applied directly onto the core, if (b) is not present, or onto the barrier coating.

In an embodiment, the invention provides pharmaceutical formulations for the modified release of niacin, including a niacin-containing core comprising a therapeutically effective amount of niacin, a salt thereof, or a niacin prodrug, a pharmaceutically acceptable release controlling agent, and one or more other pharmaceutically acceptable excipients.

In an embodiment the invention provides pharmaceutical formulations for the modified release of niacin, comprising:

(a) a niacin-containing core comprising a therapeutically effective amount of niacin, a salt thereof, or a niacin prodrug, and one or more pharmaceutically acceptable excipients;

(b) optionally, a barrier coating applied onto the core; and

(c) optionally, an enteric coating applied directly onto the core, if (b) is not present, or onto the barrier coating.

In aspects of the invention, a niacin-containing core is provided comprising niacin, a salt thereof, or a niacin prodrug, and pharmaceutically acceptable excipients. Where an immediate release core is desired, conventional pharmaceutical excipients for preparing a solid oral dosage forms will be used, such as diluents, binders, lubricants, and the like. Other such ingredients which are required for processing are also within the scope of the invention.

Where a controlled or extended release niacin-containing core is desired, a pharmaceutically acceptable release controlling material will be added. Such materials can include hydrophilic materials such as, but not limited to, sodium carboxymethylcelluloses, hydroxypropylcelluloses, hydroxyethylcelluloses, hydroxypropyl methylcelluloses, carboxymethylamide, potassium methacrylatedivinylbenzene co-polymers, polymethylmethacrylates, polyvinylpyrrolidones, polyvinylalcohols, methylcelluloses, carboxymethylcelluloses, polyoxyethylene glycols, xanthan gum, carbomers, Polyox, hydrocolloids such as natural or synthetic gums, cellulose derivatives other than those listed above, carbohydrate-based substances such as acacia, gum tragacanth, locust bean gum, guar gum, agar, pectin, carrageen, soluble alginates, carboxypolymethylene, and the like.

Hydrophobic materials which can be used include, but are not limited to, ethylcelluloses, magnesium stearate, stearic acid, hydrogenated castor oil, glyceryl monostearate, glyceryl behenate, talc, etc. Mixtures of hydrophilic and hydrophobic materials can also be used

The niacin-containing core can be in the form of niacin-containing tablets of a variety of sizes and shapes, mini-tablets, non-pareil seed materials onto which the niacin is coated, niacin pellets prepared by granulation or extrusion, together with pharmaceutically acceptable excipients, release controlling materials, and the like. The preparation of such drug-containing cores is within the scope of understanding of a person skilled in the art. The type and amounts of the release controlling materials will determine the duration of release of niacin that will be provided by the niacin-containing core, such for example an immediate release of niacin over a few minutes, or a release similar to that of the NIASPAN product, or any intermediate dissolution profile that is desired.

In embodiments, the niacin-containing core is an immediate release core with an enteric coating applied directly onto the core. In other embodiments, the niacin-containing core is an extended release core. In an embodiment, the release of niacin from the extended release niacin core is controlled using a hydrophobic material alone. In an embodiment, the hydrophobic material is a pH-independent polymeric material, such as, for example, a methacrylic acid polymer or a material such as an ethylcellulose, a wax such as carnauba wax, glyceryl monostearate, and the like. The niacin-containing core is further coated with an enteric coating. An enteric coating is provided to delay the release of the niacin from the immediate release or modified release core to allow the delivery of the niacin at a time point intermediate between that provided by immediate release formulations and extended release formulations. The provision of an enteric coating and excipients provide a formulation which generates pharmacokinetic profiles of niacin in the body upon administration to a mammal in need thereof, which are distinct from any pharmacokinetic profiles of known niacin formulations The exposure of niacin achieved in the body as measured by the area under the plasma concentration-time curves (AUC) obtained after administration of the formulations of the invention to a mammal (e.g., a dog or human), is significantly higher than that achieved upon administration of either the sustained release formulations or extended release formulations known in the art and commercially available. This is due to unique characteristics of formulations of the invention.

According to an aspect of the invention, significant pharmacokinetic advantages are provided by the inventive formulations which comprise a barrier coating interposed between the niacin-containing core and the enteric coating. Without being bound by theory, it is possible that the barrier coating prevents chemical interactions between the niacin from the core and the enteric coating material, which could result in significantly reduced release of the niacin from the formulation.

Thus, according to yet another embodiment, there are provided low dose niacin formulations comprising modified release niacin as described herein, which formulations provide statistically significant increases in Cₘₐₓ and/or AUC upon administration of the formulation to a mammal, when compared with a presently commercially available intermediate release product NIASPAN of similar strength.

In embodiments of the invention, the formulations result in a significant reduction in the dose of niacin required to provide a therapeutic effect.

Modified release pharmaceutical formulations of the present invention exhibit a slow rate of drug release as compared to immediate release formulations (formulations having an in vitro release of more than about 75% of the contained niacin in less than about 2 hours, or in about 1 hour, when tested using a customary dissolution testing method using a medium having pH values greater than about 5, such as pH 6.8 phosphate buffer, pH 7.5 phosphate buffer, etc.).

As used herein, a “therapeutically effective amount” is an amount that has an acceptable risk-to-benefit ratio for the treatment of certain diseases in the subjects in need thereof. A “therapeutically effective amount of niacin,” in the context of the present invention, includes about 250 mg, about 500 mg, about 750 mg, about 1000 mg, about 1500 mg, about 2000 mg, about 2500 mg, or about 3000 mg of niacin daily.

Niacin is extensively metabolized to produce different metabolites, the metabolism involving two pathways: pathway 1 and pathway 2.

Pathway 1 produces a nicotinuric acid (NUA) metabolite. Flushing is a significant adverse effect caused by this metabolite. the higher the levels of NUA, the greater will be the degree of cutaneous flushing.

Pathway 2 produces nicotinamide (NMA), 6-hydroxy nicotinamide (6NH), nicotinamide —N-oxide (MNO), N-methyl nicotinamide (MNA) and nicotinamide adenine dinucleotide (NAD) metabolites. Hepatotoxicity is the major adverse effect caused as a result of these metabolites.

Under in vivo conditions, pathway 2 is a high affinity, low capacity path and generates metabolites through phase I reactions. The pathway 1 metabolites are generated through a low affinity, high capacity conjugation path. The formulations of the invention can provide a plasma and urine metabolites profile which are distinct

In embodiments, the niacin from modified release formulations is released after a delay of about 10 minutes, about 20 minutes, about 30 minutes, about 1 hour, or longer after a dosage form enters into aqueous media. Depending on the particular formulation, release of niacin can occur in a pH-independent manner, or can occur only in environments having pH values at least about 5, or at least about 6. Release characteristics are determined using an in vitro procedure, where a formulation is immersed into an aqueous medium, simulating the release that would be obtained in vivo following oral administration. In some instances, the initial aqueous medium will be acidic, simulating the stomach environment, followed after a time by immersion of the formulation into a higher pH medium to simulate the gastrointestinal tract environment after stomach emptying. In vitro testing initially using an acidic medium may not accurately predict in vivo results, since emptying of the stomach occurs at varying times, due to the presence or absence of food and other factors.

Release-controlling polymers, in the context of the present invention, include hydrophilic polymers, hydrophobic polymers, delayed release (e.g., enteric) polymers, bioadhesive (or mucoadhesive) polymers, hydrophobic substances like waxes and fats, and combinations thereof. The content of release-controlling polymer in the formulations of the present invention may vary from about 1% to about 90%, or from about 5% to about 80%, of the total weight of the formulation.

Useful hydrophilic polymers of various grades include, but are not limited to: cellulose derivatives such as carboxymethyl celluloses, hydroxypropyl methylcelluloses (hypromelloses or HPMC), hydroxyethylcelluloses, hydroxypropylcelluloses (HPC), cross-linked sodium carboxymethylcelluloses, and cross-linked hydroxypropylcelluloses; carboxymethyl amides; potassium methacrylate/divinylbenzene copolymers; polyhydroxyalkyl methacrylates; polyvinylpyrrolidones (povidones or PVP) and cross-linked polyvinylpyrrolidones; high molecular weight polyvinylalcohols; gums such as natural gum, guar, agar, agarose, sodium alginate, carrageenan, fucoidan, furcellaran, laminaran, hypnea, eucheums, gum Arabic, gum ghatti, gum karaya, gum tragacanth and locust bean gum; hydrophilic colloids such as alginates; carbomers and polyacrylamides; other substances such as arbinoglactan, pectin, amylopectin, gelatin, N-vinyl lactams, polysaccharides; and the like. Combinations of any two or more of these polymers, and other polymers having the required properties are within the scope of the invention.

Useful hydrophobic polymers or combinations thereof used in various ratios include, but are not limited to: celluloses such as methylcelluloses, ethylcelluloses, cellulose acetates and their derivatives, cellulose acetate phthalate, hydroxypropyl methylcellulose phthalates, cellulose acylates, cellulose diacylates, cellulose triacylates, cellulose acetate, cellulose diacetate, cellulose triacetate, mono-, di- and tri-cellulose alkanylates, mono-, di-, and tri-cellulose arylates, and mono-, di- and tri-cellulose alkenylates; crosslinked vinylpyrrolidone polymers (crospovidone); polymethacrylic acid based polymers and copolymers such as are sold by Evonik Industries AG, Essen, Germany as EUDRAGIT™ (including Eudragit RL and RS, NE 30 D, and NM 30 D); zein; and aliphatic polyesters. Other classes of polymers, copolymers of these polymers or their mixtures in various ratios and proportions are within the scope of this invention without limitation.

EUDRAGIT NE 30 D and NM 30 D products are 30% aqueous dispersions of polymers having the repeating unit (A), and are called “Polyacrylate Dispersion 30 Per Cent” in the European Pharmacopeia. The NE 30 D polymer has an average molecular weight about 800,000 and the NM 30 D polymer has an average molecular weight about 600,000.

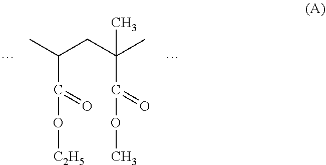

An enteric coating is a coating that prevents release of an active agent until the dosage form reaches a pH environment higher than that of the stomach. A delayed release dosage form comprises niacin and is coated with an enteric polymer. The enteric polymer should be non-toxic and is predominantly soluble in intestinal fluids, but substantially insoluble in the gastric juices. Examples of such delayed release (enteric) polymers include polyvinylacetate phthalates (PVAP), hydroxypropyl methylcellulose acetate succinates (HPMCAS), cellulose acetate phthalates (CAP), methacrylic acid copolymers, hydroxypropyl methylcellulose succinates, cellulose acetate succinates, cellulose acetate hexahydrophthalates, hydroxypropyl methylcellulose hexahydrophthalates, hydroxypropyl methylcellulose phthalates (HPMCP), cellulose propionate phthalates, cellulose acetate maleates, cellulose acetate trimellitates, cellulose acetate butyrates, cellulose acetate propionates, methacrylic acid/methacrylate polymers (acid number 300 to 330 and also known as EUDRAGIT L), which are anionic copolymers based on methacrylate and available as a powder (also known as methacrylic acid copolymer, type A NF, methacrylic acid-methyl methacrylate copolymer, ethyl methacrylate-methylmethacrylate-chlorotrimethylammonium ethyl methacrylate copolymer, and the like), and combinations comprising one or more of the foregoing enteric polymers. Other examples include natural resins, such as shellac, copal collophorium, and combinations comprising one or more of the foregoing polymers. Yet other examples of enteric polymers include synthetic resins bearing carboxyl groups. The methacrylic acid-acrylic acid ethyl ester 1:1 copolymer sold under the trade designation EUDRAGIT L 100-55 is suitable. This polymer is the solid present in an aqueous dispersion sold as EUDRAGIT L 30 D-55.

EUDRAGIT L 100-55 is designated “Methacrylic Acid-Ethyl Acrylate Copolymer (1:1), Type A” in the European Pharmacopeia. It has the repeating units of (B) and an average molecular weight about 250,000.

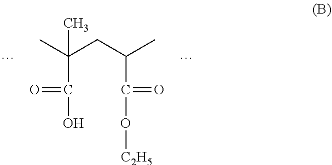

A barrier coating may be optionally applied to a core formulation to prevent interactions between the drug and enteric coating. It can also impart moisture protection to the core formulation. Non limiting examples of barrier coating materials include hydrophilic and hydrophobic excipients such as hydroxypropyl methylcelluloses (hypromellose or HPMC), hydroxypropylcelluloses, ethyl cellulose and other cellulose derivatives, polyvinyl acetates, polyvinyl alcohols, sugars, amino acids, zein, polyvinylpyrrolidones, guar gum, etc. Other inert materials, which can act as a barrier to prevent any interaction between the niacin and the enteric or functional polymer, are within the scope of the invention without limitation. The determination of the thickness of the barrier coating as well as the viscosity grade of a polymeric material, if used, are within the understanding of a person skilled in the art. Thus, when a polymeric material such as a HPMC is used, a suitable grade could include a low viscosity grade capable of acting as a barrier between the niacin and the enteric coating material without impacting the dissolution and release of the niacin upon contact with an aqueous medium. When a sugar is used as a barrier coating, the thickness of the coating will determine the degree of protection that such a coating will provide, as also will the type of sugar. Such and other aspects of selection of a barrier coating are within the scope of understanding of a person skilled in the art of preparation of solid oral dosage forms.

Other materials which can be used to prevent undesired interactions between the niacin-containing core and the enteric coating include acidic materials such as for example citric acid, ascorbic acid, tartaric acid, benzoic acid, and amino acids, such as, for example, aspartic acid, and glutamic acid. Other materials which can provide an acidic environment and prevent interaction between niacin and the enteric coating are also included within the scope of the invention without limitation. The acidic stabilizing materials can be blended with the ingredients before the niacin-containing core is compressed, or the materials can be layered onto a niacin-containing core to prevent interaction between the niacin-containing core and the enteric coating. The acidic materials can be used with niacin in weight ratios of 0.01:1 to 0.5:1, acidic material to niacin.

Any barrier material that is used and, whatever the mechanism by which the barrier coat acts to prevent the undesired interaction between the niacin-containing core and the enteric coat may be, the surprisingly high exposure levels provided by the inventive formulation will follow. Any such barrier coating is thus within the scope of the invention without limitation.

A bioadhesive polymer may be included in oral dosage forms to increase the contact time between the dosage form and the mucosa of a drug-absorbing section of the gastrointestinal tract. Non-limiting examples of bioadhesives include carbomers (various grades), sodium carboxymethylcelluloses, methylcelluloses, polycarbophils (e.g., NOVEON™), hydroxypropyl methylcelluloses, hydroxypropyl celluloses, sodium alginate, sodium hyaluronate, and combinations comprising two or more of the foregoing.

Hydrophobic substances such as waxes and fats may have a melting point of about 30° C. to about 200° C., or about 45° C. to about 90° C. Useful hydrophobic substances can include neutral or synthetic waxes, fatty alcohols (such as lauryl, myristyl, stearyl, cetyl or cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, and combinations comprising two or more of the foregoing materials. Suitable waxes include beeswax, paraffin wax, carnauba wax, etc., and also synthetic waxes such as for example microcrystalline waxes and other commercially available waxes, castor wax, and wax-like substances, e.g., materials normally solid at room temperature and having a melting point of about 30° C. to about 100° C., and combinations comprising two or more of the foregoing waxes.

Of course, any other release-controlling polymers, which demonstrate similar characteristics, are also acceptable in the working of this invention.

In some embodiments of the present invention, pharmaceutically acceptable excipients serving as pharmaceutically inert cores comprise: insoluble inert materials, such as glass particles/beads or silicon dioxide, calcium phosphate dihydrate, dicalcium phosphate, calcium sulfate dihydrate, microcrystalline cellulose (MCC) or cellulose derivatives; soluble cores such as acid cores like tartaric acid and sugar spheres of sugars like dextrose, lactose, anhydrous lactose, spray-dried lactose, lactose monohydrate, mannitol, starches, sorbitol, sucrose; insoluble inert polymeric materials such as spherical or nearly spherical core beads of polyvinyl chloride, polystyrene, or any other pharmaceutically acceptable insoluble synthetic polymeric material; and the like and mixtures thereof.

Modified release formulations comprising niacin and a release-controlling polymer may be prepared by any suitable technique including those described below. The active agent and a release-controlling polymer may, for example, be prepared by wet granulation techniques, melt extrusion techniques, etc. The active agent in modified release formulations can include a plurality of substrates comprising the active ingredient, which substrates are coated with a sustained-release coating comprising a release-controlling polymer. The modified release formulations may thus be made in conjunction with a multiparticulate system, such as beads, ion-exchange resin beads, spheroids, microspheres, seeds, pellets, granules, and other multiparticulate systems in order to obtain a desired modified (delayed-extended) release of the active agent. The multiparticulate system can be presented in a tablet or capsule or other suitable unit dosage form. In certain cases, more than one multiparticulate system can be used, each exhibiting different characteristics, such as pH dependence of release, time for release in various media (e.g., acidic, basic, simulated intestinal fluids), release in vivo, size, and formulation.

In some cases, a spheronizing agent, together with the active ingredient, can be spheronized to form spheroids. Microcrystalline cellulose and hydrous lactose impalpable are examples of such agents. Additionally (or alternatively), the spheroids can contain a water insoluble polymer, such as an acrylic polymer, an acrylic copolymer, such as a methacrylic acid-ethyl acrylate copolymer, or an ethyl cellulose. In this formulation, the release-modifying coating will generally include a water insoluble material such as a wax, either alone or in admixture with a fatty alcohol, or shellac or zein. Spheroids or beads, coated with an active ingredient can be prepared, for example, by dissolving or dispersing the active ingredient in a solvent and then spraying the solution onto a substrate, for example, sugar spheres NF-21, 18/20 mesh, using a Würster insert. Optionally, additional ingredients are also added prior to coating the beads in order to enhance the active ingredient binding to the substrates, and/or to color the resulting beads, etc. The resulting substrate-active material may optionally be over-coated with a barrier material, to separate the therapeutically active agent from the next coating of material, e.g., a release-controlling polymer.

The pharmaceutical formulations of the present invention can be prepared by various other methods and techniques as known to the skilled person so as to achieve desired in vitro drug release profiles. Specific embodiments of processes comprise any of:

1. Direct compression, using appropriate punches and dies, the punches and dies being fitted to a suitable rotary tableting press.

2. Injection or compression molding using suitable molds fitted to a compression unit.

3. Granulation followed by compression.

4. Extrusion in the form of a paste, into a mold or into an extrudate to be cut into desired lengths.

When particles are made by direct compression, the addition of lubricants may be helpful and sometimes this is important to promote powder flow and to prevent capping of the compressed particle (breaking off of a portion of the particle) when compression pressure is relieved. Typically, lubricants are added in a concentration of from 0.25% to 3% by weight. Additional excipients may be added to enhance powder flowability and reduce adherence.

Oral dosage forms may be prepared to include an effective amount of melt-extruded subunits in the form of multi-particulates within a capsule. For example, a plurality of the melt-extruded multi-particulates can be placed in a gelatin capsule in an amount sufficient to provide an effective release dose when ingested and contacted by gastric fluid. The subunits, e.g., in the form of multi-particulates, can be compressed into oral tablets using conventional tableting equipment using standard techniques.

The formulations may be in the form of microtablets enclosed inside a capsule, e.g., a gelatin capsule. For this, any gelatin capsule employed in the pharmaceutical formulation field can be used, such as the hard gelatin capsules known as CAPSUGEL™, available from Pfizer.

In an embodiment, pharmaceutical formulations of the present invention can be prepared using a granulation process comprising:

a) dissolving or dispersing the active ingredient optionally with binder and/or solubilizer in a solvent;

b) granulating the pharmaceutically acceptable excipient blend with the solution comprising active;

c) drying and lubricating the granules; and

d) compressing the granules into tablets, or alternatively filling into capsules.

In another embodiment, pharmaceutical formulations of the present invention can be prepared using a direct compression process comprising:

a) mixing the active ingredient and a release-controlling polymer, optionally with other pharmaceutically acceptable excipients; and

b) compressing the blend of a) into tablets, or alternatively filling into capsules.

Alternatively, the formulations of the present invention can be prepared by dissolving the active ingredient in a suitable solvent, and layering the dissolved active, optionally with other excipients, onto the surface of inert cores such as tartaric acid and the like as described above. Such drug-layered cores or pellets may further be granulated or coated with a release-controlling polymer to produce pharmaceutical formulations of the present invention.

The granules/beads or tablets or capsules may further be coated with a release-controlling polymer, optionally with other excipients. Such coating can be done using various known techniques such as dip coating, pan coating, fluidized bed coating, and the like.

The residual solvent content of the pharmaceutical formulations, as described herein, may be made low, such as less than about 5000 ppm by weight. The concentration of residual solvents can further be reduced to desired limits, such as are acceptable by regulatory authorities, such as using drying steps.

Surfactants/solubilizers that may be useful in the formulations of the present invention include, but are not limited to: anionic surfactants like potassium laurate, sodium lauryl sulfate, sodium dodecylsulfate, alkyl polyoxyethylene sulfates, sodium alginate, dioctyl sodium sulfosuccinate, phosphatidyl choline, phosphatidyl glycerol, phosphatidyl inosine, phosphatidylserine, phosphatidic acid and their salts, glyceryl esters, sodium carboxymethylcellulose, cholic acid and other bile acids (for example, cholic acid, deoxycholic acid, glycocholic acid, taurocholic acid and glycodeoxycholic acid) and salts thereof (for example, sodium deoxycholate); cationic surfactants like quaternary ammonium compounds (e.g., benzalkonium chloride, cetyltrimethylammonium bromide, lauryldimethylbenzylammonium chloride, acyl carnitine hydrochlorides and alkyl pyridinium halides); nonionic surfactants like polyoxyethylene fatty alcohol ethers (MACROGOL™ and BRIJ™), polyoxyethylene sorbitan fatty acid esters (polysorbates or TWEEN™), polyoxyethylene fatty acid esters (MYRJ™), sorbitan esters (SPAN™), glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), polaxamines, and the like; and mixtures thereof.

In the context of the present invention, during the processing of the pharmaceutical formulations into finished dosage forms, one or more pharmaceutically acceptable excipients may optionally be used, including but not limited to: diluents such as microcrystalline cellulose (“MCC”), silicified MCC (e.g., PROSOLV™), microfine cellulose, lactose, starch, pregelatinized starches, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide, and the like; cores/beads such as insoluble inert materials like glass particles/beads or silicon dioxide, calcium phosphate dihydrate, dicalcium phosphate, calcium sulfate dihydrate, microcrystalline cellulose, cellulose derivatives; soluble cores such as sugar spheres of sugars like dextrose, lactose, mannitol, starches, sorbitol, or sucrose; insoluble inert polymeric materials such as spherical or nearly spherical core beads of polyvinyl chloride, polystyrene or any other pharmaceutically acceptable insoluble synthetic polymeric material, and the like or mixtures thereof; binders or adherents such as acacia, guar gum, alginic acid, dextrin, maltodextrin, methylcelluloses, ethylcelluloses, hydroxyethyl celluloses, hydroxypropyl celluloses (e.g., KLUCEL®), hydroxypropyl methylcelluloses (e.g., METHOCEL®), carboxymethyl cellulose sodium, povidones (various grades of KOLLIDON®, PLASDONE®), starches and the like; disintegrants such as carboxymethyl cellulose sodium (e.g., Ac-Di-Sol®, PRIMELLOSE®), crospovidones (e.g., KOLLIDON®, POLYPLASDONE®), povidone K-30, polacrilin potassium, starches, pregelatinized starches, sodium starch glycolate (e.g. EXPLOTAB®), and the like; plasticizers such as acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, oleyl alcohol, myristyl alcohol and the like. Solvents that may be used in formulation processing include, for example, water, methanol, ethanol, isopropyl alcohol, acetone, methylene chloride, dichloromethane, and the like, and mixtures thereof.

Pharmaceutical formulations of the present invention may further include any one or more of pharmaceutically acceptable glidants, lubricants like sodium stearyl fumarate, opacifiers, colorants, and other commonly used excipients.

The pharmaceutical formulations of the present invention exhibit desired in vivo absorption profiles for drugs delivered. The in vivo pharmacokinetic parameters frequently used to evaluate pharmaceutical formulations after oral administration include maximum plasma concentration (“Cₘₐₓ”), time after administration until the maximum plasma concentration (“Tₘₐₓ”), area under the plasma concentration-time plot curve (“AUG”), and the like.

The pharmaceutical formulations of the invention may contain one or more active ingredients in addition to niacin. Non-limiting examples of such additional active ingredients include lipid lowering agents, anti-diabetic compounds, NSAIDs, cox-2 inhibitors, PGD2 antagonists to control the flushing, anti-arrhythmic agents, anti-coagulants, anti-depressants, anti-hypertensive agents, α-glucosidase inhibitors, immunosuppressants, sedatives, hypnotics, beta-blockers, cardiac ionotropic agents, corticosteroids, diuretics, anti-anginal agents, muscle relaxants, nutritional agents, opioid analgesics, muscle relaxants, cognition enhancers, cholesterol absorption inhibitors, bile acid sequestering agents, and the like. Typically, lipid lowering compounds include statins, fibrates and PPAR agonists. Exemplary statins include atorvastatin, simvastatin, lovastatin, pravastatin, cervastatin, fluvastatin, while fibrates comprise fenofibrate, gemfibrozil, and bezafibrate. Non-limiting examples of DPP IV inhibitors include sitagliptan, vildagliptan, saxagliptan. Typical anti-diabetic compounds include sulfonylureas, meglitinides, DPP-IV inhibitors, biguanides, peroxisome proliferator activated receptor (“PPAR”) agonists, glucose uptake modulators. Cholesterol absorption inhibitors include ezetimibe, and the like. Bile acid sequestering agents include orlistat, and the like.

The pharmaceutical formulations disclosed herein can be advantageously used for the treatment of hyperlipidemia, hypercholesterolemia and mixed dyslipidemia, myocardial infarction, atherosclerotic diseases, and other such conditions that respond to treatment with niacin.

The following examples are provided to illustrate certain specific aspects and embodiments of the invention and demonstrate the practice and advantages thereof. It is to be understood that the examples are given for purposes of illustration only and are not intended to limit the scope of the invention in any manner.

### Examples 1-2

### Delayed-Extended Release Formulations Comprising Niacin

mg/Tablet

| Ingredient | 1 | 2 |
|---|---|---|
| Niacin | 500 | 500 |
| Microcrystalline cellulose | 50 | 50 |
| (Avicel ™ PH101)** | | |
| Anhydrous lactose | 50 | 50 |
| Eudragit ™ NM 30 D | 16 | 16 |
| Croscarmellose sodium | 6.5 | 5 |
| Stearic acid | 5 | 6.5 |
| Hydroxypropyl methylcellulose (HPMC) 6 cps | — | 12 |
| Isopropyl alcohol‡ | — | 25 |
| Water‡ | — | 10 |
| Eudragit L 100-55 | 50.2 | 51.2 |
| Isopropyl alcohol‡ | 86 | 86 |
| Hydroxypropyl methylcellulose (HPMC) 6 cps | 12 | — |
| Isopropyl alcohol‡ | 25 | — |
| Water‡ | 10 | — |
| Meloxicam | 2.5 | — |
| Hydroxypropyl methylcellulose (HPMC) 6 cps | 50 | — |
| Isopropyl alcohol‡ | 102 | — |
| Water‡ | 41 | — |

*Eudragit products are co-polymers of methacrylic acid and methacrylates and are products of Evonik Industries AG, Germany.
**Avicel products are products of FMC Biopolymer Inc.
‡Evaporates during processing.

Manufacturing process:

1. Niacin, microcrystalline cellulose and anhydrous lactose are mixed together and passed through a BSS #60 mesh sieve.

2. The mixture from 1 is again blended in a blender to attain uniformity.

3. The blend of 2 is granulated in a rotating mixer granulator (RMG) using a Eudragit NM 30 D dispersion.

4. The wet granules from 3 are dried and passed through a BSS #24 mesh sieve.

5. Croscarmellose sodium is passed through a BSS #60 mesh sieve and mixed with granules from 4.

6. Stearic acid is passed through a BSS #60 mesh sieve and mixed with granules from 5.

7. The blended granules of 6 are compressed into tablets using 19×8 mm punches.

Barrier coating for Example 2:

8. HPMC 6 cps is dispersed in a mixture of isopropyl alcohol and water, with stirring.

9. The dispersion of 8 is coated onto the tablets produced in 7. Enteric and final coating:

10. Eudragit L 100-55 solution is prepared in isopropyl alcohol with stirring.

11. The core tablets of Example 1 and barrier coated tablets of Example 2 are coated with Eudragit L 100-55 solution of step 10, to produce an 8% by weight increase.

12. HPMC 6 cps is dissolved in a mixture of isopropyl alcohol and water, and coated onto the enteric coated Example 1 tablets from 11, to produce a 2% weight gain.

13. Meloxicam and HPMC 6 cps are dissolved in a mixture of isopropyl alcohol and water, and coated onto tablets of step 12).

14. HPMC 6 cps (final quantity) is dissolved in a mixture of isopropyl alcohol and water, and coated onto the tablets from 13.

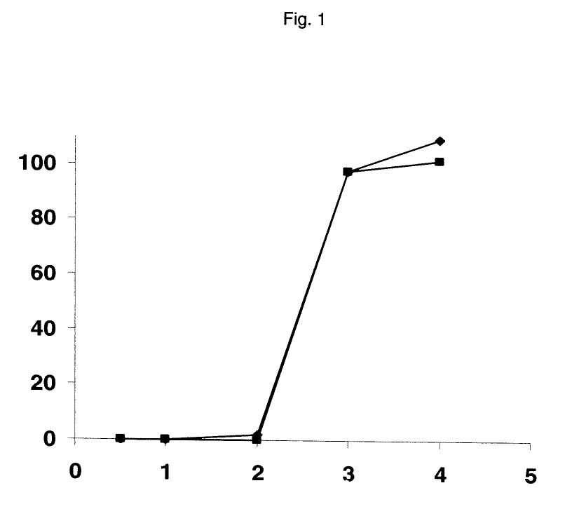

In vitro release profiles of niacin from the formulations of Examples 1 and 2 are determined using 0.1 N hydrochloric acid for an initial 2 hours, followed by phosphate buffer pH 6.8, using apparatus 2 (paddle) and 75 rpm stirring, with the procedure of Test 711 “Dissolution” in United States Pharmacopeia 29, United States Pharmacopeial Convention, Inc., Rockville, Md., 2005 (“USP”). The dissolution profile results are illustrated in FIG. 1, where the vertical axis is cumulative percent of contained niacin that dissolved and the horizontal axis is hours; Example 1 data points are represented using the diamond symbol and Example 2 data points are represented using the square symbol.

Formulations prepared according to Examples 1 and 2 are administered orally to four healthy male beagle dogs under fasting conditions for evaluating the pharmacokinetic parameters of niacin. Blood samples are withdrawn periodically over 72 hours after administration and niacin concentration in the plasma is analyzed. The results are below.

| Mean Plasma Value | Example 1 | Example 2 |
|---|---|---|
| Cₘₐₓ (ng/mL) | 538 | 35550 |
| AUG₍₀₋ₜ₎ (ng · hour/mL) | 8867 | 72549 |
| Tₘₐₓ (hours) | 2.5 | 1.83 |

The results indicate that the presence of a barrier coating affects systemic exposure to niacin, provided by the formulation when administered to a mammal.

### Examples 3-8

### Delayed-Extended Release Formulations Comprising Niacin

| | mg/Tablet | | | | | |
|---|---|---|---|---|---|---|
| Ingredient | 3 | 4 | 5 | 6 | 7 | 8 |
| Niacin | 500 | 500 | 500 | 500 | 500 | 500 |
| Microcrystalline cellulose | 280 | 50 | 50 | 50 | — | 50 |
| (Avicel PH101) | | | | | | |
| Microcrystalline cellulose | — | — | — | — | 50 | — |
| (Avicel PH112) | | | | | | |
| Anhydrous lactose | — | 50 | 50 | 25 | — | — |
| Tabletose ™ 70 | — | — | — | — | 50 | — |
| Croscarmellose sodium | 25 | 5 | 6.5 | — | 5 | — |
| Eudragit NM 30 D | — | 16 | 16 | 16 | 16 | 13.75 |
| Stearic acid | — | 6.5 | 5 | 6 | 6.5 | 5.63 |
| Eudragit L100-55 | 50 | — | 50 | — | — | — |
| Triethyl citrate | 12 | — | — | — | — | — |
| Isopropyl alcohol‡ | 86.5 | — | 86 | — | — | — |
| HPMC 6 cps | — | — | 12 | — | 12.5 | — |
| Isopropyl alcohol‡ | — | — | 25 | — | 25 | — |
| Water‡ | 37.5 | — | 10 | — | 10 | — |
| Meloxicam | — | — | 2.5 | — | — | — |
| HPMC 6 cps | — | — | 50 | — | — | — |
| Isopropyl alcohol‡ | — | — | 70 | — | — | — |
| Water‡ | — | — | 30 | — | — | — |
| HPMC 6 cps | — | — | 15 | — | — | — |
| Isopropyl alcohol‡ | — | — | 32 | — | — | — |
| Water‡ | — | — | 10 | — | — | — |
| Talc | 8.7 | — | — | — | — | — |
| Eudragit L 100-55 | — | — | — | — | 51 | — |
| Isopropyl alcohol‡ | — | — | — | — | 860 | — |

‡Evaporates during processing.

Manufacturing process for Example 3:

1. A blend of niacin, microcrystalline cellulose and croscarmellose sodium is passed through a BSS #60 mesh sieve, mixed in a blender, then is compressed into tablets using 19×8 mm punches.

Coating

2. Eudragit L 100-55 solution is prepared in isopropyl alcohol and water with stirring. Triethyl citrate and talc are added, with stirring.

3. Core tablets of 1 are coated with the solution of 2, to produce an 8% weight increase.

Manufacturing process for Examples 4-8:

1. Niacin, microcrystalline cellulose and anhydrous lactose are mixed together and passed through a BSS #60 mesh sieve.

2. The powder mixture is again blended in a blender to attain uniformity.

3. The blend of 2 is granulated in a rapid mixer granulator (RMG) using a Eudragit NM 30 D dispersion.

4. Wet granules from 3 are dried and passed through a BSS #24 mesh sieve.

5. Stearic acid is passed through a BSS #60 mesh sieve and mixed with granules from 4.

6. The blended granules of 5 are compressed into tablets using 19×8 mm punches.

Coating

7. A Eudragit L 100-55 solution is prepared in isopropyl alcohol with stirring. Triethyl citrate is added, where required.

8. Core tablets of 6 are coated with the solution of 7, to produce an 8% weight increase.

9. HPMC 6 cps is dissolved in a mixture of isopropyl alcohol and water, and coated onto the tablets from 8, to produce a 2% weight gain.

10. For Example 5, meloxicam and HPMC 6 cps (second quantity) are dissolved in isopropyl alcohol and water, and coated onto tablets from 9.

11. For Example 5, HPMC 6 cps (third quantity) is dissolved in isopropyl alcohol and water, and coated onto tablets from 10, to produce a 2% weight gain.

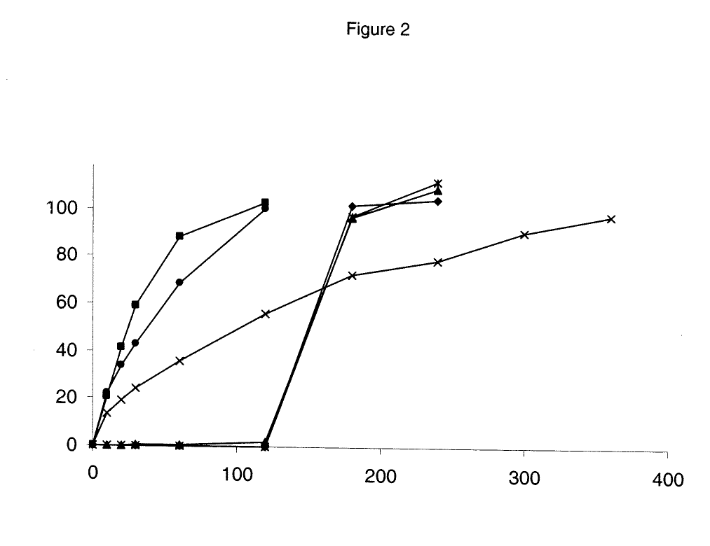

In vitro release profiles of niacin from the formulations of Examples 3, 5, and 7 are determined using the conditions and procedure described for Examples 1 and 2. In vitro release profiles of niacin from the formulations of Examples 4, 6, and 8 are determined using the same conditions and procedure described for Examples 1 and 2, except that the medium used is only 0.001 N HCl (pH 3.0). The results are illustrated in FIG. 2, where the vertical axis is cumulative percent of contained niacin that dissolved and the horizontal axis is minutes; Example 3 data points are represented using the diamond symbol, Example 4 data points are represented using the square symbol, Example 5 data points are represented using the triangle symbol, Example 6 data points are represented using the “x” symbol, Example 7 data points are represented using the asterisk symbol, and Example 8 data points are represented using the dot symbol.

### Example 9

### Extended Release Formulation Comprising Niacin 500 mg

| Ingredient | mg/Tablet |
|---|---|
| Niacin | 500 |
| Microcrystalline cellulose | 50 |
| (Avicel PH112) | |
| Lactose monohydrate | 50 |
| Eudragit NM 30 D | 32 |
| Croscarmellose sodium | 5 |
| Stearic acid | 6.5 |
| HPMC 6 cps | 19.3 |
| Isopropyl alcohol‡ | 38.6 |
| Water‡ | 15.5 |
| HPMC phthalate | 63.52 |
| Isopropyl alcohol‡ | 256 |
| Water‡ | 102 |
| Triethyl citrate | 9.07 |
| Sodium bicarbonate | 20.17 |
| HPMC 6 cps | 22.66 |
| Isopropyl alcohol‡ | 45.2 |
| Water‡ | 26.6 |

‡Evaporates during processing.

Manufacturing process:

1. Niacin, microcrystalline cellulose and lactose monohydrate are mixed together and passed through a BSS #60 mesh sieve.

2. The powder mixture is again blended in a blender to attain uniformity.

3. The blend of 2 is granulated in an RMG using a Eudragit NM 30 D dispersion.

4. The wet granules from 3 are dried and passed through a BSS #24 mesh sieve.

5. Croscarmellose sodium and stearic acid are passed through a BSS #60 mesh sieve and mixed with granules from step 4.

6. The blended granules of 5 are compressed into tablets using 19×8 mm punches.

Coating

7. A sub-coating solution is prepared by dispersing HPMC 6 cps (first quantity) in isopropyl alcohol and water, with stirring until a clear solution formed.

8. The coating solution obtained from 7 is coated onto tablets prepared in 6.

9. An enteric coating solution is prepared by dispersing HPMC phthalate, triethyl citrate, and sodium bicarbonate in a mixture of isopropyl alcohol and water, with stirring.

10. Sub-coated tablets from 8 are coated with coating solution of 9.

11. HPMC 6 cps (second quantity) is dissolved in isopropyl alcohol and water, and coated onto the enteric coated tablets of 11 to produce a 2% weight gain.

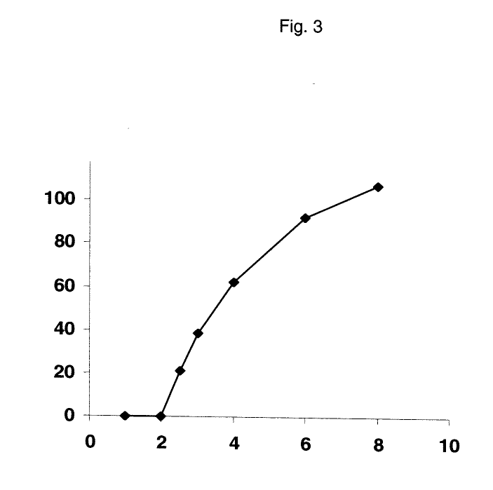

An in vitro release profile of niacin from the formulation of Example 9 is determined using conditions similar to those of Examples 1 and 2. The results are illustrated in FIG. 3, where the vertical axis is cumulative percent of contained niacin that dissolved and the horizontal axis is hours.

### Examples 10-11

### Extended Release Formulations Comprising Niacin 500 mg

mg/Tablet

| Ingredient | 10 | 11 |
|---|---|---|
| Niacin | 500 | 500 |
| Microcrystalline cellulose | 50 | 50 |
| (Avicel PH112) | | |
| Lactose monohydrate | 50 | 50 |
| Eudragit NM 30 D | 32 | 16 |
| Croscarmellose sodium | 5 | 5 |
| Stearic acid | 6.5 | 6.5 |
| HPMC 6 cps | 19.3 | — |
| Opadry ™ Yellow 03B82626* | — | 18.82 |
| Isopropyl alcohol‡ | 38.6 | — |
| Water‡ | 15.5 | 54.1 |
| HPMC phthalate | 63.52 | 26.55 |
| Isopropyl alcohol‡ | 256 | 107 |
| Water‡ | 102 | 42.5 |
| Triethyl citrate | 9.07 | 3.79 |
| Sodium bicarbonate | 20.17 | 8.43 |
| Opadry Yellow 03B82626 | — | 37.68 |
| HPMC 6 cps | 22.66 | — |
| Isopropyl alcohol‡ | 45.2 | — |
| Water‡ | 26.6 | 120 |

*Opadry Yellow is a formulated coating product from Colorcon, containing hydroxypropyl methylcellulose, titanium dioxide, macrogol, talc, and iron oxide yellow.
‡Evaporates during processing.

The manufacturing process is similar to that of Example 9, except that Opadry Yellow is used for coating instead of the HPMC in steps 7, 8 and 11 as described in the manufacturing process of Example 9.

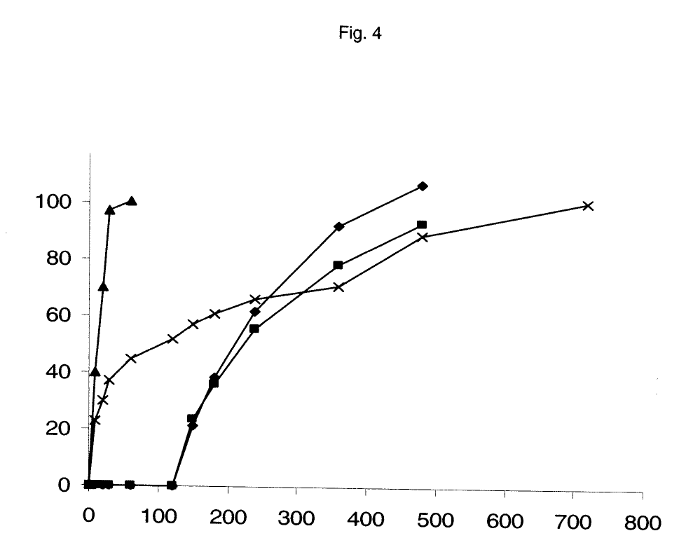

In vitro release profiles of niacin from the products of Examples 10 and 11, the commercial NIACOR product, and the commercial NIASPAN product, each containing 500 mg of niacin, are determined using conditions similar to those for Examples 1 and 2. The results are given below and profiles are illustrated in FIG. 4, where the vertical axis is cumulative percent of contained niacin that dissolved and the horizontal axis is minutes; Example 10 data points are represented using the diamond symbol, Example 11 data points are represented using the square symbol, NIACOR data points are represented using the triangle symbol, and NIASPAN data points are represented using the “x” symbol.

| | Cumulative % of Niacin Dissolved | | | |
|---|---|---|---|---|
| MINUTES | EXAMPLE 10 | EXAMPLE 11 | NIACOR | NIASPAN |
| 0 | 0 | 0 | 0 | 0 |
| 10 | 0 | 0 | 40 | 23 |
| 20 | 0 | 0 | 70 | 30 |
| 30 | 0 | 0 | 97 | 37 |
| 60 | 0 | 0 | 100 | 45 |
| 120 | 0 | 0 | — | 52 |
| 150 | 21 | 24 | — | 57 |
| 180 | 38 | 36 | — | 61 |
| 240 | 62 | 56 | — | 66 |
| 360 | 92 | 79 | — | 71 |
| 480 | 107 | 93 | — | 89 |
| 720 | — | — | — | 101 |

1. A pharmaceutical formulation comprising:
(a) a niacin-containing non-swellable solid core comprising a therapeutically effective amount of niacin, a salt thereof, or a niacin prodrug, a pharmaceutically acceptable release controlling agent, and at least one pharmaceutically acceptable excipient;
(b) optionally, a barrier coating over the niacin-containing core; and
(c) optionally, an enteric coating applied onto either (a) or (b).

2. A pharmaceutical formulation according to claim 1, wherein a release controlling agent comprises a methacrylic acid copolymer.

3. A pharmaceutical formulation according to claim 1, wherein a release controlling agent comprises a methacrylic acid copolymer having repeating unit (A).

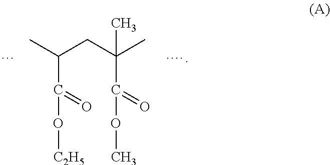

4. A pharmaceutical formulation according to claim 3, wherein a methacrylic acid copolymer has a molecular weight about 600,000.

5. A pharmaceutical formulation according to claim 3, wherein a methacrylic acid copolymer has a molecular weight about 800,000.

6. A pharmaceutical formulation according to claim 1, wherein a core comprises niacin, a salt thereof, or a niacin prodrug, and a diluent, granulated with a pharmaceutically acceptable release controlling agent.

7. A pharmaceutical formulation according to claim 1, wherein a barrier coating is present and comprises a hydrophilic polymer.

8. A pharmaceutical formulation according to claim 1, wherein a barrier coating is present and comprises a hydroxypropyl methylcellulose.

9. A pharmaceutical formulation according to claim 1, wherein less than about 50 percent of contained niacin is released within about 2 hours, following immersion into an aqueous medium having pH values less than about 4.

10. A pharmaceutical formulation according to claim 1, wherein less than about 25 percent of contained niacin is released within about 2 hours, following immersion into an aqueous medium having pH values less than about 4.

11. A pharmaceutical formulation according to claim 1, wherein less than about 10 percent of contained niacin is released within about 2 hours, following immersion into an aqueous medium having pH values less than about 4.

12. A pharmaceutical formulation, comprising:
(a) a niacin-containing core comprising a therapeutically effective amount of niacin, a salt thereof, or a niacin prodrug, and a pharmaceutically acceptable excipient, granulated with a pharmaceutically acceptable release controlling agent;
(b) optionally, a barrier coating layered onto the core; and
(c) an enteric coating applied directly onto the core, if (b) is not present, or onto the barrier coating.

13. A pharmaceutical formulation according to claim 12, wherein a release controlling agent comprises a methacrylic acid copolymer.

14. A pharmaceutical formulation according to claim 12, wherein a release controlling agent comprises a methacrylic acid copolymer having repeating unit (A).

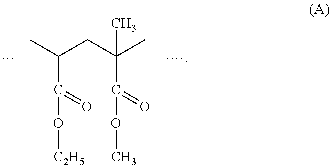

15. A pharmaceutical formulation according to claim 14, wherein a methacrylic acid copolymer has a molecular weight about 600,000.

16. A pharmaceutical formulation according to claim 14, wherein a methacrylic acid copolymer has a molecular weight about 800,000.

17. A pharmaceutical formulation according to claim 12, wherein a barrier coating is present and comprises a hydroxypropyl methylcellulose.

18. A pharmaceutical formulation comprising niacin in a core and a polymer coating over the core, wherein less than about 50 percent of contained niacin is released within about 2 hours, following immersion into an aqueous medium having pH values less than about 4.

19. A pharmaceutical formulation according to claim 18, wherein less than about 25 percent of contained niacin is released within about 2 hours, following immersion into an aqueous medium having pH values less than about 4.

20. A pharmaceutical formulation according to claim 18, wherein less than about 10 percent of contained niacin is released within about 2 hours, following immersion into an aqueous medium having pH values less than about 4.
